(19) United States
(12) Patent Application Publication
(10) Pub. No.: US 2022/0049238 A1
(43) Pub. Date: Feb. 17, 2022

(54) Synthetic Matrix Assembled and Rapidly Templated Spheroids, Organoids and 3D Cell Cultures

(71) Applicant: Rutgers, The State University of New Jersey, New Brunswick, NJ (US)

(72) Inventors: Ki-Bum Lee, Monmouth Junction, NJ (US); Christopher Rathman, Piscataway, NJ (US); Letao Yang, Piscataway, NJ (US)

(73) Assignee: Rutgers, The State University of New Jersey, New Brunswick, NJ (US)

(21) Appl. No.: 17/404,030

(22) Filed: Aug. 17, 2021

Related U.S. Application Data
(60) Provisional application No. 63/066,440, filed on Aug. 17, 2020.

(51) Int. Cl. C12N 11/14; A61L 27/02; A61L 27/36; A61L 27/54; A61L 27/58; A61K 35/30

(52) U.S. Cl. CPC C12N 11/14; A61L 27/025; A61L 27/3604; A61L 2430/38; A61L 27/58; A61L 27/3675; A61K 35/30; A61L 27/54

(57) ABSTRACT

Disclosed are spheroidal hybrid biodegradable materials containing low dimensional manganese dioxide (MnO₂) support structures and cells, methods of manufacture thereof, and methods of use thereof.

### CROSS-REFERENCE TO RELATED APPLICATION

The present application claims the benefit of priority under 35 U.S.C. § 119(e) of U.S. Provisional Application Ser. No. 63/066,440, filed Aug. 17, 2020. The entire disclosure of the application noted above is incorporated herein by reference.

### FIELD OF THE INVENTION

The field of the invention is directed to biodegradable low dimensional manganese dioxide-based hybrid cell spheroid inorganic materials for delivery of cells and therapeutic agents, and methods of manufacture and use thereof.

### BACKGROUND

Developing reliable therapeutic methods to treat central nervous system (CNS) diseases (e.g. Alzheimer's and Parkinson's diseases), degeneration in the aging brain, and CNS injuries (e.g. spinal cord injury (SCI) and traumatic brain injuries), is a current major challenge, due to the complex and dynamic cellular microenvironment during the disease progression. Current therapeutic approaches aim to restore neural signaling, reduce neuro-inflammation, and prevent subsequent damage to the injured area, using stem cell therapies. However, there are many current limitations with using stem cell therapies. Due to the inflammatory nature of the injured regions, most of the cells perish soon after transplantation. Additionally, the extracellular matrix (ECM) of the damaged areas is not conducive to stem cell survival and differentiation. Accordingly, there is a need for better approaches to increase the survival rate of transplanted stem cells, and to better control stem cell fate in vivo, which can lead to the recovery of the damaged neural functions and the repair of neuronal connections in a more effective manner.

Nanoscaffolding-based techniques and scaffold-free 3D cell spheroid techniques have been used in the art, and both have advantages and disadvantages associated with them. The four main types of scaffolds in the art include hydrogels, polymeric scaffolds, micropatterened surface microplates, and nanofiber based scaffolds. Most of these scaffolds, however, once used for transplantation purposes, will burst release therapeutic drugs, which negatively influences the survival or differentiation of the cells transplanted. Additionally, conventional scaffolds are cumbersome to seed stem cells at high densities, do not offer the ability for tissue self-assembly, and their transplantation in vivo can require invasive procedures. Three-dimensional (3D) cell culture systems are becoming increasingly popular due to their ability to mimic tissue-like structures at high cellular densities, effectively modeling the embryonic stages. The scaffold-free 3D cell spheroids created by conventional 3D culture systems offer the advantages of low initial cell seeding density, the possibility of tissue self-assembly, and they also enable the injectable delivery of high density stem cells at the sites of injury or disease, thereby promoting functional recovery. However, disadvantageously, cells in the core of scaffold-free 3D cell spheroids often go through stress, due to insufficient oxygen and nutrient diffusion, which results in cell apoptosis after long-term culture, also known as “necrotic core”. Additionally, there are limited cell types that can be used to form spheroids using conventional techniques. As such, there is an urgent need for improved solutions that mimic the natural environment for therapeutic purposes, for example, for disease modeling, drug screening, cell therapy or tissue engineering. In particular, there is a need for a structure that offers a combination of at least some of the benefits associated with both nanoscaffolding and non-scaffold-based 3D spheroid techniques. Additionally, there is a need for a 3D cell culture technique for generating at least one of spheroids or organoids, that overcome at least one of the disadvantages associated with existing nanoscaffolding or scaffold-free 3D spheroid techniques.

### SUMMARY OF THE DISCLOSURE

The present disclosure relates to biodegradable scaffolding material that possesses a number of surprising therapeutic benefits and uses, as well as methods of making and using the nanoscaffolding material.

Accordingly, in a first aspect of the invention, there is provided a biodegradable scaffolding material containing a plurality of at least one of zero-dimensional, one-dimensional or two-dimensional manganese dioxide support structures, and a plurality of cells, wherein the zero-dimensional, one-dimensional or two-dimensional manganese dioxide support structures each have a surface, and upon each surface is a coating containing a plurality of cell adhesion molecules; wherein the zero-dimensional, one-dimensional or two-dimensional manganese dioxide support structures define a structure containing a plurality of interstices, and the plurality of cells are disposed around and between the zero-dimensional, one-dimensional or two-dimensional manganese dioxide support structures and through the zero-dimensional, one-dimensional or two-dimensional manganese dioxide support structure interstices; wherein the cell adhesion molecules contain a plurality of cell binding domains, have a binding affinity with the zero-dimensional, one-dimensional or two-dimensional manganese dioxide support structures, and promote the adhesion of the cells to the zero-dimensional, one-dimensional or two-dimensional manganese dioxide support structures; and wherein together, the support structures and the plurality of cells are configured to assemble to form at least one spheroid.

According to some embodiments of the disclosure, the cell adhesion molecules contain at least one of biopolymers or small molecules, wherein the small molecules have at least one of a mass of no more than 900 daltons, a molecular weight of no more than 1500, or a size of from about 0.5 nm to about 1.5 nm. According to some embodiments, the cell binding domains are peptides. According to some embodiments, the peptides conatin at least one of an arginine-glycine-aspartic acid (RGD) amino acid sequence, a cationic amine group, an amphiphilic unit, or a combination thereof. According to some embodiments, the peptides contain at least one linear arginine-glycine-aspartic acid (RGD) amino acid sequence. According to some embodiments, the zero-dimensional, one-dimensional or two-dimensional manganese dioxide support structures contain at least one of, 0-dimensional nanoparticles, 1-dimensional manganese dioxide nanotubes, 1-dimensional manganese dioxide nanorods, or 2-dimensional manganese dioxide nanosheets. According to some embodiments, the cells are stem cells, endoderm cells, mesoderm cells, ectoderm cells, cancer cells, or a combination thereof. According to some embodiments, the stem cells are neural stem cells, mesenchymal stem cells, induced pluripotent stem cells (iPSCs), or a combination thereof. According to some embodiments, the scaffolding material further contains at least one agent. According to some embodiments, the agent is a therapeutic agent. According to some embodiments, the therapeutic agent is a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, diagnostic composition, psychiatric medication, psychoactive compound, or a combination thereof.

In a second aspect of the invention, there is provided a method of making a biodegradable scaffolding material including providing at least one of a plurality of zero-dimensional, one-dimensional or two-dimensional manganese dioxide support structures; applying a plurality of cell adhesion molecules including a plurality of cell binding domains to a surface of the zero-dimensional, one-dimensional or two-dimensional manganese dioxide support structures; and mixing the zero-dimensional, one-dimensional or two-dimensional manganese dioxide support structures with a cell suspension containing a plurality of cells; so that the support structures, cell adhesion molecules and cells assemble in the suspension to form a mixture containing a plurality of spheroids. According to some embodiments, the mixture is cultured for a sufficient period of time for the spheroids to form at least one organoid. According to some embodiments, the plurality of cell adhesion molecules are applied to the surface of the zero-dimensional, one-dimensional or two-dimensional manganese dioxide support structures by a method of coating; spraying; or self-assembly via at least one of electrostatic, hydrophobic or hydrophilic interactions, van der waals interactions, or hydrogen bonding; or a combination thereof. According to some embodiments, the cells are stem cells, endoderm cells, mesoderm cells, ectoderm cells, cancer cells, or a combination thereof. According to some embodiments, the stem cells are neural stem cells, mesenchymal stem cells, induced pluripotent stem cells (iPSCs), or a combination thereof. According to some embodiments, the size of the spheroid or organoid is tuned by controlling at least one of the ratio of cells to zero-dimensional, one-dimensional or two-dimensional manganese dioxide support structures, the concentration of the cells, or the concentration of the zero-dimensional, one-dimensional or two-dimensional manganese dioxide support structures. According to some embodiments, the method further includes the step of loading at least one agent onto the zero-dimensional, one-dimensional or two-dimensional manganese dioxide support structures. According to some embodiments, the agent is a therapeutic agent. According to some embodiments, the therapeutic agent is a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, diagnostic composition, psychiatric medication, psychoactive compound, or a combination thereof. According to some embodiments, the rate of delivery of the therapeutic agent is controlled by tuning the rate of degradation of the zero-dimensional, one-dimensional or two-dimensional manganese dioxide support structure. According to some embodiments, the spheroids are formed within a period of time of from 1 to 5 minutes. According to some embodiments, the at least one organoid is formed within a period of time of no more than 24 hours. According to some embodiments, the rate of degradation of the zero-dimensional, one-dimensional or two-dimensional manganese dioxide support structure is tuned by controlling at least one of, the porosity of the scaffolding material, thickness of the scaffolding material, the aspect ratio of the scaffolding material, or cell density. According to some embodiments, the rate at which the biodegradable scaffolding material is degraded in vivo can be measured by detecting the rate of release of Mn⁺² ions from the biodegradable scaffolding material. According to some embodiments, the zero-dimensional, one-dimensional or two-dimensional manganese dioxide support structures include at least one of, 0-dimensional nanoparticles, 1-dimensional manganese dioxide nanotubes, 1-dimensional manganese dioxide nanorods, or 2-dimensional manganese dioxide nanosheets. According to some embodiments, the cell adhesion molecules include at least one of biopolymers or small molecules, wherein the small molecules have at least one of a mass of no more than 900 daltons, a molecular weight of no more than 1500, or a size of from about 0.5 nm to about 1.5 nm. According to some embodiments, the cell binding domains are peptides. According to some embodiments, the peptides include at least one of an arginine-glycine-aspartic acid (RGD) amino acid sequence, a cationic amine group, an amphiphilic unit, or a combination thereof. According to some embodiments, the method further includes the step of applying a vacuum filtration method to the resultant mixture to isolate the biodegradable scaffolding material. According to some embodiments, the method further includes the step of centrifuging the resultant mixture prior to applying the vacuum filtration method.

In a third aspect of the invention, there is provided a biodegradable scaffolding material made according to the method of the second aspect of the disclosure.

In a fourth aspect of the invention, there is provided a method of treating a disease or disorder in a subject, including the step of surgically implanting or injecting the biodegradable scaffolding material according to the first aspect of the disclosure into a subject.

### BRIEF DESCRIPTION OF THE DRAWINGS

FIG. 1A presents a schematic diagram of the proposed mechanism for the self-assembly of cells with cell-binding domain-functionalized 2-dimensional manganese dioxide nanosheets, to form a hybrid biodegradable nanoscaffolding material spheroid; FIG. 1B shows a magnetic resonance image demonstrating that biodegradation of the low dimensional manganese dioxide support structures can be monitored by Magnetic Resonance Imaging (MRI).

FIG. 2 shows a schematic diagram of advanced stem cell therapy for enhanced treatment of spinal cord injury (SCI) by the injection of spheroidal nanoscaffolding material into a mouse model having a damaged spinal cord, resulting in the the differentiation of the cells in the spheroids into functional neurons, providing for spinal cord regeneration and functional recovery.

FIG. 3 displays the Basso Mouse Scale (BMS) scores of injured mice transplanted with neural cells, conventional spheroids (“Neurosphere”) or the 3D hybrid biodegradable nanoscafflolding material (“SMART Neurosphere”), demonstrating the superior functional recovery of mice transplanted with the nanoscafflolding material, as compared to those transplanted with conventional spheroids.

FIG. 4 shows an image of a nanoscaffold material that has formed a beating organoid that was formed from cardiac tissue.

FIGS. 5A and 5B present the release of drugs from the nanoscaffold material spheroid caused by the controllable degradation of the low dimensional manganese dioxide support structures; FIG. 5A shows a schematic diagram of release of a drug from a nanoscaffold spheroid at a moment in time; FIG. 5B displays a series of fluorescent images showing the release of a drug in a sustainable and monitorable manner from nanoscaffold material spheroids over a period of three days.

FIG. 6 shows a schematic diagram of the growth and conglomeration of spheroids in culture, which over time create organoids.

### DETAILED DESCRIPTION

The present disclosure relates to biodegradable hybrid inorganic scaffolding materials including low dimensional manganese dioxide support structures and a plurality of cells that assemble to form 3D spheroids. Conventional scaffolds, such as graphene-based nanoscaffolds, synthetic polyurethanes, hydrogels, polymeric scaffolds, micropatterened surface microplates, and nanofiber based scaffolds, offer advantages such as good mechanical properties and the ability to functionalize with biomolecules. However, they suffer from disadvantages such as high initial cell density and lack of ability for tissue self-assembly. Conventional three-dimensional culture systems, such as liquid overlay, hanging drop, hydrogel embedding, spinner flask bioreactor, scaffold and three-dimensional bioprinting, provide 3D cell cultures having advantages such as low initial cell density and the possibility of tissue self-assembly. However, they suffer from disadvantages such as poor mechanical properties and the inability to functionalize with biomolecules. The hybrid scaffolding material of the present disclosure includes both scaffolding and 3D cell features, and thus offers several advantages over traditional strategies based solely on scaffolding techniques or scaffold-free 3D cell techniques. These advantages include at least one of, generalized cell assembly, fast cell assembly, high cell survival rates including under inflammatory or toxic conditions, improved differentiation, improved control over spheroid formation, and improved cell behavior in the spheroids. The scaffolding material of the present disclosure thus have a potential for use in many applications, such as at least one of, disease modeling, drug target identification, tissue engineering, drug or gene delivery, stem cell therapy, or regenerative medicine. In particular, with regard to stem cell therapy, applications can include treatment of SCI and other nervous system injuries/disorders (e.g. central nervous system (CNS) and peripheral nervous system (PNS) injuries/disorders), treatment of orthopedic injuries/disorders (e.g. bone and cartilage), dental, cardiac disease, and/or muscular injuries/disorders, amongst others.

The biodegradable scaffolding material of the invention contains a plurality of low dimensional manganese dioxide support structures, and a plurality of cells. The term “low dimension” or “low dimensional” structures denotes structures that are lower in dimension than 3D, viz., 0-dimensional, 1-dimensional (1D), 2-dimensional (2D), or any combination thereof. The surface of the low dimensional manganese dioxide support structures is at least partially coated with a plurality of cell adhesion molecules. The term “partially coated” denotes coverage of at least about 25%, more preferably at least about 30%, more preferably at least about 40%, most preferably at least about 50% of the surface. In some embodiments, the surface of the low dimensional manganese dioxide support structures is substantially covered with the cell adhesion molecules. The term “substantially covered” denotes coverage of between about 50% and about 90% of the surface. In some embodiments, the surface of the low dimensional manganese dioxide support structures is fully covered with the cell adhesion molecules. The term “fully covered” denotes coverage of at least about 90%, preferably at least about 95%, more preferably at least about 99%, most preferably 100% of the surface. The cell adhesion molecules have a binding affinity to the low dimensional manganese dioxide support structures. Additionally, the cell adhesion molecules have moieties, “cell binding domains,” attached thereto, which have a binding affinity to the cells in the scaffolding material. Thus, the cell adhesion molecules provide cell binding functionalization to the low dimensional manganese dioxide support structures, thereby promoting the adhesion of the cells to the low dimensional manganese dioxide support structures, and enabling multiple cells to bind to each low dimensional manganese dioxide support structure. One cell can simultaneously recruit multiple units of cell binding domains. Together, the assembly of cells and cell-binding domain functionalized low dimensional manganese dioxide support structures assemble to form at least one spheroid, where the low dimensional manganese dioxide support structures function as building blocks for the spheroids.

The biodegradable scaffolding material can include only 0D, only 1D, or only 2D manganese dioxide support structures, or any combination thereof. In some embodiments of the disclosure, the plurality of low dimensional manganese dioxide support structures includes 2-dimensional nanosheets. In some embodiments of the invention, the nanosheets are 2D MnO₂ nanosheets. However, other 2-dimensional metal oxide compositions can be suitable for use in the present invention. Generally, suitable metal oxides include transition metals with a +4 oxidation state bound to two oxygen atoms. Other 2D metal oxides beyond 2D MnO₂ that can be suitable include, for example, vanadium (IV) dioxide (VO₂), for example as disclosed in Chem. Commun., 2013, 49, 3943-3945; cobalt (IV) peroxide (CoO₂) for example as disclosed in J. Power Sources, 227, 101-105; and nickel (IV) peroxide (NiO₂), for example as disclosed in Langmuir, 2014, 30(47), 14343-14351; each reference hereby incorporated by reference in their entireties. Within the 2D MnO₂ nanosheets are a plurality of interstices. The plurality of cells surround the nanosheets, and also infiltrate the nanosheet interstices, such that they are disposed on all sides of the nanosheets, and also pass through the nanosheets by way of the nanosheet interstices. The cells have a binding affinity with the nanosheets, due to the presence of the cell adhesion molecules on the nanosheets, which promote adhesion of the cells to the manganese dioxide support structures. This affinity causes the nanosheets to self-assemble themselves in such a way as to create at least one spheroid, creating the scaffolding material.

In another embodiment of the invention, the plurality of low dimensional manganese dioxide support structures includes 1-dimensional nanotubes and/or nanorods. In some embodiments of the invention, the nanotubes or nanorods are 1D MnO₂ nanotubes or nanorods. However, other 1-dimensional metal oxide compositions can be suitable for use in the present invention. Generally, suitable metal oxides include the same as those listed herein above for the nanosheets. The cells have a binding affinity with the 1D nanotubes and nanorods, due to the presence of the cell adhesion molecules on the nanotubes and nanorods, which causes the nanotubes or nanorods to self-assemble themselves into at least one spheroid, creating the scaffolding material. Between the 1D nanotubes and nanorods in the lattice are a plurality of interstices. The plurality of cells surround the 1D nanotubes and nanorods in the lattice, and also infiltrate the lattice interstices, such that they are disposed on all sides of the 1D nanotubes and nanorods, and also pass through the lattice interstices.

In a further embodiment, the plurality of low dimensional manganese dioxide support structures include 0-dimensional nanoparticles. In some embodiments of the invention, the nanoparticles are 0D MnO₂ nanoparticles. However, other 0-dimensional metal oxide compositions can be suitable for use in the present invention. Generally, suitable metal oxides include the same as those listed herein above for the nanosheets. The cells have a binding affinity with the 0D nanoparticles, due to the presence of the cell adhesion molecules on the nanoparticles, which causes the nanoparticles to self-assemble themselves into at least one spheroid, creating the scaffolding material. Between the 0D nanoparticles in the lattice are a plurality of interstices. The plurality of cells surround the 0D nanoparticles in the lattice, and also infiltrate the lattice interstices, such that they are disposed on all sides of the 0D nanoparticles, and also pass through the lattice interstices.

There is no limit to the number of low dimensional manganese dioxide support structures that can be used in a nanoscaffold material of the disclosure, as long as the support structures remain on the nano level. The support structures can be situated in any arrangement, relative to one another. They can be arranged substantially parallel to one another, in a way that they are substantially planar, in a random arrangement, or in any combination of the foregoing.

As noted above, the cell adhesion molecules have a binding affinity with the low dimensional manganese dioxide support structures. While not being bound by any particular theory it is believed that the cell adhesion molecules interact (i.e. associate with) with the low dimensional manganese dioxide support structures through intramolecular forces, such as electrostatic interactions and metal-π interactions. These type of interactions typically involve interactions with amine and aromatic functional groups (e.g. through phenylalanine, tyrosine, tryptophan, asparagine, glutamine, lysine, arginine, or histidine amine groups and aromatic sidechains on cell adhesion molecules) and the oxygen and manganese atoms respectively. It is surmised that the amine groups interact with oxygen via electrostatic interactions, while the π-systems of the aromatic groups interact with the Mn in the nanoscaffolds. Other potential interactions could include, for example, but not necessarily, ionic bonding, hydrogen bonding, Van der Waals forces, dipole-dipole interactions, dipole-induced forces, London dispersion forces, aromatic ring interactions, hydrophobic interactions, and combinations thereof. It is further surmised that the interactions between the low dimensional manganese dioxide support structures, along with the interactions between the cells and the functional groups on the cell adhesion molecules, drive the assembly of the hybrid spheroidal-shaped nanoscaffold material.

The cell adhesion molecules can be any molecules having a binding affinity to the low dimensional manganese dioxide support structures, and including moieties having a binding affinity to the cells. Examples of suitable cell adhesion molecules include biopolymers and small molecules. In some embodiments, the cell adhesion molecules are biopolymers. By “biopolymer” is meant a natural polymer that is produced by the cells of living organisms, or a synthetic polymer that is a human-made copy of a natural biopolymer. Suitable biopolymers include polypeptides (such as for example, ECM proteins, such as, for example, laminins, fibronectins, vitronectins, collagens, fibrillin, lumican, thrombospondin-4, dematophontin, basement membrane, and combinations thereof, polypeptides, such as, polylysine, polyarginine, polytyrosine, polycystein, polyhistidine, polyalanine, polytyrosine, polyvaline, polyleucine, polyserine, polysaccharides (such as for example ECM polysaccharides, such as for example, hyaluronic acid, alginate, chitosan, gellan, dextran, cellulose, curdlan, zooglan, succinoglycan, and combinations thereof, polynucelotides (such as for example polyadenines, polythymines, polygytosines, polyguanines, polyruacils). In some embodiments, the cell adhesion molecules are short peptides including from 2 to about 100 amino acids, that are derived from proteins, and mimic the function of natural proteins. Examples of suitable short peptides include, for example, RGD (arginine-glycine-aspartic acid) peptides, laminin peptides, collagen peptides, fibronectin peptides, integrin-binging peptides or a combination thereof. In some embodiments, the cell adhesion molecule is an ECM protein having an RGD (arginine-glycine-aspartic acid) cell binding domain. In some embodiments, the cell adhesion molecules are small molecules. By “small molecules” is meant non-peptidic, non-oligomeric organic compounds, either synthesized or found in nature. Small molecules are typically characterized in that they possess one or more of the following characteristics: several carbon-carbon bonds, multiple stereocenters, multiple functional groups, at least two different types of functional groups, and a molecular weight of less than 1500, although not all, or even multiple, of these features need to be present. In some embodiments, the small molecules have a molecular weight of no more than 1500. In some embodiments, the small molecules have a mass of no more than 900 daltons. In some embodiments, the small molecules have a size of from about 0.5 nm to about 1.5 nm. In some embodiments, the small molecules have a size of from about 0.75 to about 1.25 nm. In some embodiments, the small molecules have a size of about 1 nm. In some embodiments, the small molecules have at least one of a mass of no more than 900 daltons, a molecular weight of no more than 1500, or a size of from between about 0.5 nm and about 1.5 nm. In some embodiments the cell adhesion molecules are biopolymers and small molecules.

The cell binding domains can be any moiety having a binding affinity to cells. In some embodiments, the cell binding domains are peptides. In some embodiments, the peptides include at least one RGD amino acid sequence. In some embodiments, the RGD amino acid sequence is a linear RGD sequence. In some embodiments, the peptides have at least one cationic amine group. In some embodiments, the peptides have at least one amphiphilic unit. In some embodiments, the peptides have at least one of an arginine-glycine-aspartic acid (RGD) amino acid sequence, or a cationic amine group, an amphiphilic unit.

The biodegradable nanoscaffolds of the present disclosure also include cells. Any suitable cells can be used, such as for example, stem cells, endoderm cells, mesoderm cells, ectoderm cells, cancer cells, or any combination of the foregoing. In some embodiments, the cells are stem cells, such as for example, any of embryonic stem (ES) cells, adult stem cells, induced pluripotent stem (iPS) cells, induced somatic stem cells (iSC) and combinations thereof. More specifically, the stem cells can include, for example, hematopoietic stem cells (HSCs), mammary stem cells, intestinal stem cells, mesenchymal stem cells (MSCs), endothelial stem cells, neural stem cells (NSC), olfactory adult stem cells, neural crest stem cells, testicular cells, adipose-derived stem cells (ADSCs), and combinations thereof. In some embodiments, the stem cells are neural stem cells (NSCs), e.g. for treatment of spinal cord injury (SCI). In some embodiments, the cells are endoderm cells, such as for example lung cells, thyroid cells, or pancreatic cells. In some embodiments, the cells are mesoderm cells, such as for example, cardiac muscle cells, skeletal muscle cells, smooth muscle cells, tubule cells, red blood cells, bone cells or chondrocytes. In some embodiments, the cells are ectoderm cells, such as for example, epidermal skin cells, neural cells or pigment cells. In some embodiments, the cells are cancer cells, such as for example bladder, bone, breast of cervical cancer cells. In some embodiments, the cells are a combination of two or more different types of cells, which can be from the same, or different germ layers.

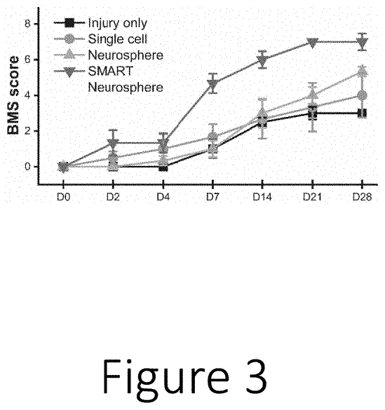

A further advantage of the scaffolding material of the present disclosure is that the scaffolding material can promote stem cell differentiation while embedded in the scaffolding material. For example, nanoscaffolding materials containing laminin promote differentiation of neural cells, which are useful for treatment of spinal cord injury (SCI), as illustrated by the Examples below and FIG. 3, demonstrating that mice having spinal injuries had superior BMS scores when treated with the present scaffolding material (“SMART Neurosphere”), compared to mice treated with conventional spheroids (“Neurosphere”). Those containing fibronectin promote myogenesis (differentiation of muscle cells) and osteogenesis (differentiation of bone cells). One of ordinary skill in the art guided by the present specification will recognize that there are a number of cell adhesion molecules, including but not limited to those disclosed herein, which result in different stem cell differentiation. The nanoscaffolding materials can thus be used for autologous grafting, e.g. autologous nerve grafting, allografting, or even xenografting.

The scaffolding material can be used for the delivery of one or more agents, such as for example therapeutic agents. In some embodiments of the disclosure, the biodegradable nanoscaffolding material includes one or more agents, such as for example at least one therapeutic agent. One or more of the therapeutic agents can be trapped, or embedded in the nanoscaffolding material. The therapeutic agents can bind to, or associate with the nanoscaffolding material. While not being bound by any particular theory it is believed that this association occurs through interactions similar to that of the cell adhesion molecules with the manganese dioxide in the nanoscaffolding. The therapeutic agents can include, but are not limited to, any therapeutic agents that contain amine and/or aromatic functional groups/side chains. Such compositions are known to one of ordinary skill in the art. For example, therapeutic agents can include, but are not limited to, any of peptides, proteins, antibodies, nucleic acids, biologic drugs, small molecules, cytokines, ligands, and combinations thereof. Other potential therapeutic agents can include, purely by way of example, chemotherapeutic agents, antipyretics, analgesics/anesthetics, antibiotics, antiseptics, hormones, stimulants, depressants, statins, beta blockers, anticoagulants, antivirals, anti-fungals, anti-inflammatories, growth factors, vaccines, diagnostic compositions, psychiatric medications/psychoactive compounds, and any related compositions.

In addition to being biodegradable, the rate of biodegradation of the slow dimensional support structures in the scaffolding material is tunable. Low dimensional manganese dioxide support structures degrade in the presence of cell metabolism outputs, such as ascorbic acid, according to a classic reduction-oxidation mechanism. In vivo, the main mechanism for controlling the rate of degradation of the low dimensional manganese dioxide support structures is the porosity of the scaffolding material. The rate of degradation of the low dimensional manganese dioxide support structures can also be controlled by other means, such as for example, controlling the thickness of the nanoscaffolding material, the aspect ratio (height to surface area ratio) of the nanoscaffolding material, the cell adhesion molecule concentration, or the cellular density. In some embodiments, the rate of biodegradation of the nanoscaffolding material is tunable, by any of these means. In some embodiments, it is tunable by changing the porosity of the nanoscaffolding material. Likewise, the rate at which the therapeutic agent or cells are released is also tunable. This is due to the fact that, the rate at which the therapeutic agent or cells are released from the biodegradable nanoscaffolding material is typically substantially equivalent to the rate at which the low dimensional support structures are degraded in vivo. For example, the low dimensional MnO₂ support structures of the present disclosure can be controlled to degrade rapidly, with full degradation, for example, in about three days, or slowly, for example, with around 20% degradation after 2 weeks. This wide range tunable and therapeutic relevant degradation profile illustrates the utility of the scaffolding material for transplanting stem cells to treat central nervous system injuries, as well as for tissue engineering in general. For example, without being bound by any particular theory, it is believed that fast degradation is not beneficial for treating spinal cord injury (SCI). On the other hand, regarding cell transplantations, a slow biodegradability is known to restrict cell migration and proliferation, and lead to nutrient and oxygen deficiencies for cells, in which case fast degradation is desired. Accordingly, the nanoscaffold materials of the present disclosure can be used for rationally guided drug selection and scaffold design, optionally using computer simulations (for example, DFT (density-functional theory) simulations) This ability to tune the rate of biodegradation, and thus the rate of release of therapeutic agents and cells, offers an advantage, for example, over existing technologies, which do not biodegrade. The ability to control biodegradability by tuning scaffolding material properties, instead of relying solely on in vivo redox conditions, is advantageous because in vivo redox environments are difficult to control.

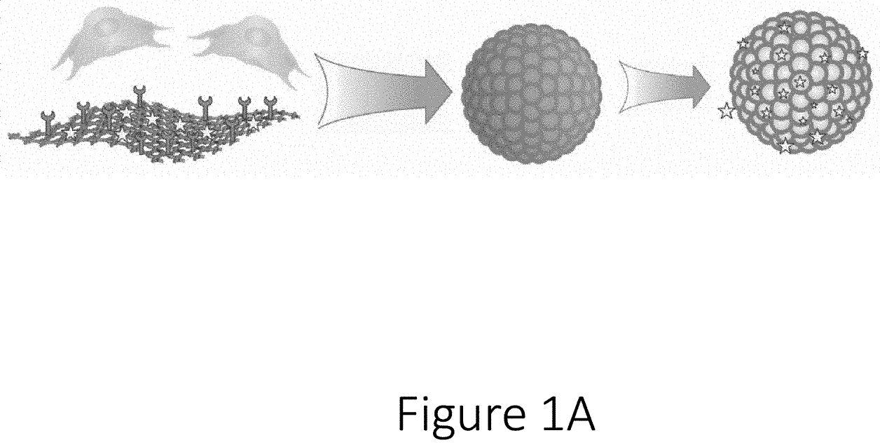

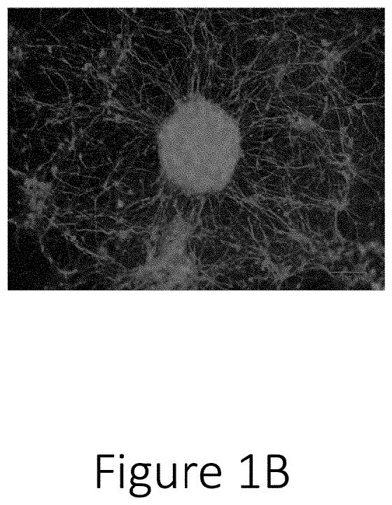

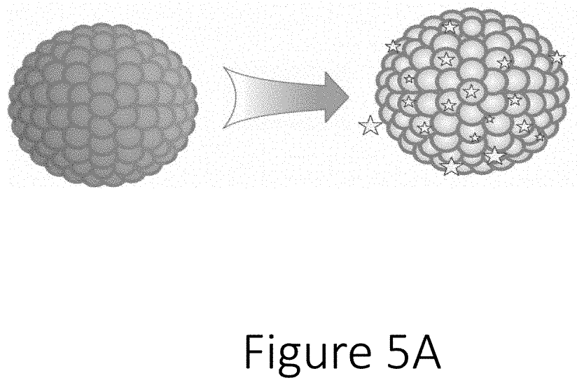

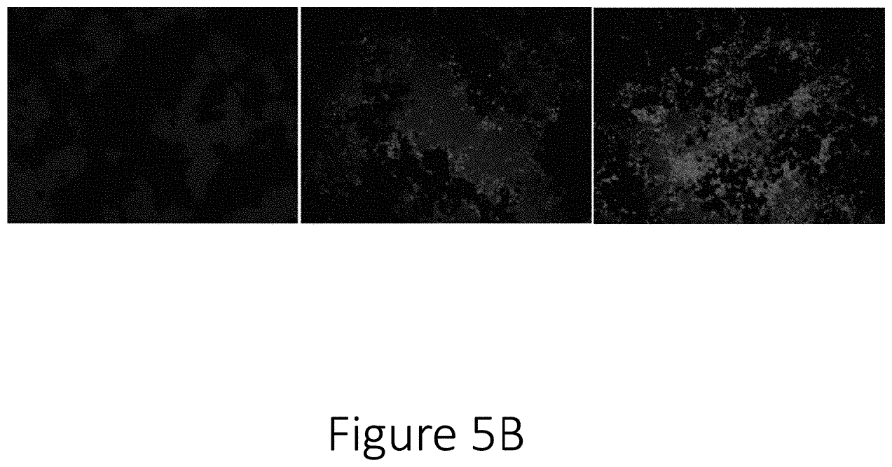

Another advantage of the scaffolding material is that the release of agents from the scaffolding material can be monitored by standard imaging techniques known in the art. The rate at which the biodegradable low dimensional MnO₂-containing support structures degrade in vivo can be measured by detecting the release of Mn⁺² ions from the scaffolding material (for example by MRI (magnetic resonance imaging) or FRET (Fluorescence Resonance Energy Transfer). The low dimensional MnO₂-containing support structures release Mn⁺² on degradation, producing an MRI-detectable signal (see e.g. FIG. 1B) which can be used to quantify the degradation rate. As noted above, the rate at which the therapeutic agent or cells are released from the nanoscaffolding material, is typically substantially equivalent to the rate at which the low dimensional MnO₂-containing support structures are degraded in vivo. Thus, the rate at which the therapeutic agent is released is measurable, by quantifying the rate/amount of Mn⁺² released. Additionally, because Mn+2 is similar to Ca+2, it can be internalized by cells and retained, rather than being cleared immediately. Low dimensional MnO₂ support structures also serve as fluorescent quenchers and enable detection of degradation by drug release visualized with FRET (see e.g. FIG. 5A, with drug represented by stars; and FIG. 5B, fluorescence increase over time confirming release of drug by biodegradation of the MnO₂ support structure).

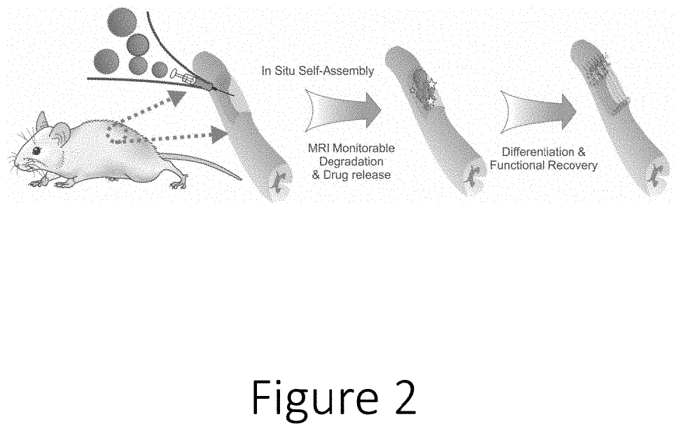

The nanoscaffold material can be used in a variety of in vivo model systems, providing at least one of, improved viability, drug delivery, biocompatibility, or control of differentiation, after insertion of the nanoscaffold material into an in vivo environment. The nanoscaffold material of the present disclosure can be used to treat, or prevent a disease or disorder in a subject in need thereof. In some embodiments of the disclosure, the nanoscaffold material is surgically implanted, for example by grafting or inserting, into the subject. In a different embodiment, the nanoscaffolds are injected into the subject. See e.g. FIG. 2 where nanoscaffolding material containing a drug (represented by stars) is injected into the mouse having a spinal cord injury. In Situ self-assembly of the injected nanoscaffolding material into spheroids and/or organoids with concomitant drug release allows differentiation of the cells into viable tissue, thereby providing functional recovery from the spinal cord injury. Whether implanted or injected, the nanoscaffolding material would typically contain at least one therapeutic agent, such as those described hereinabove. The diseases or disorders that the nanoscaffold material of the present disclosure can be used to treat are explicitly not limited. The examples presented herein show treatment of spinal cord injury (SCI), however, this is only one possible application. Other diseases/disorders include any of congenital disorders, neurological disorders, muscular disorders, metabolic disorders, autoimmune disorders, cellular proliferative disorders, e.g. neoplasms or cancers, viral infections, bacterial infections, protist infections, fungal infections, acute tissue injuries/trauma, chronic tissue injuries/trauma, and combinations thereof. Explicitly non-limiting examples of diseases/disorders for which the nanoscaffold materials can be used in treatment include, abdominal aortic aneurysm, acne, acute cholecystitis, acute lymphocytic leukemia (ALL), acute myeloid leukemia (AML), acute pancreatitis, Addison's disease, alcoholism, allergic rhinitis, Alzheimer's disease, anal cancer, angioedema, ankylosing spondylitis, anorexia nervosa, arthritis including rheumatoid arthritis, asthma, atopic eczema, bile duct cancer, bipolar disorder, bladder cancer, blood cancer, blood poisoning, bone cancer, bone marrow cancer, bowel cancer, bowel polyps, brain stem disorders, brain tumors, breast cancer, bronchiectasis, bronchitis, bursitis, burns, cellulitis, cervical cancer, chest infections, chronic kidney disease, chronic pancreatitis, chronic myeloid leukemia (CML), chronic obstructive pulmonary disease (COPD), clostridium difficile infection, congenital heart diseases, costochondritis, Crohn's disease, cystic fibrosis, cystitis, deep vein thrombosis (DVT), dementia with Lewy bodies, dental abscesses, diabetes (Type I and II), diabetic retinopathy, diverticulitis, erectile dysfunction, Ewing sarcoma, fibroids, fibromyalgia, gallbladder cancer, ganglion cysts, germ cell tumors, hairy cell leukemia, head and neck cancer, heart failure, hearing loss, hepatitis A, B, and C, hyperlipidemia, high cholesterol, HIV/AIDS, Hodgkin lymphoma, Non-Hodgkin lymphoma, hyperglycemia, hypoglycemia, hyperhidrosis, idiopathic pulmonary fibrosis, iron deficiency anemia, irritable bowel syndrome (IBS), Kaposi's sarcoma, kidney cancer, kidney failure, kidney infection, labyrinthitis, Langerhans cell histiocytosis, laryngeal cancer, liver cancer, liver disease, liver tumors, lung cancer, lupus, Lyme disease, malaria, malignant brain tumors, meningitis, mesothelioma, migraines, multiple myeloma, multiple sclerosis (MS), nasal and sinus cancer, nasopharyngeal cancer, neuroblastoma, neuroendocrine tumors, non-alcoholic fatty liver disease (NAFLD), obesity and related disorders, organ failure, osteoarthritis, osteoporosis, osteosarcoma, ovarian cancer, ovarian cysts, overactive thyroid disorders, pancreatic cancer, Parkinson's disease, penile cancer, peripheral neuropathy, pneumonia, polymyalgia rheumatic, prostate cancer, psoriasis, psoriatic arthritis, psychiatric disorders, reactive arthritis, retinoblastoma, rhabdomyosarcoma, rosacea, septic shock, sexually transmitted infections (STIs), sickle cell disease, Sjogren's syndrome, skin cancers, spinal cord injury (SCI), stomach cancer, testicular cancer, thyroid cancer, ulcerative colitis, vaginal cancer, vulvar cancer, Wilms tumor, any related disorders, and combinations thereof.

The scaffolding material can be made by any suitable method. In some embodiments, the scaffolding material is made by providing a plurality of zero-dimensional, one-dimensional or two-dimensional manganese dioxide support structures, or any combination thereof. Cell adhesion molecules, which are described hereinabove, are applied to the surface of the plurality of low dimensional manganese dioxide support structures so that at least some of the low dimensional support structures are coated with cell adhesion molecules. In some embodiments, either substantially all, or all of the low dimensional manganese dioxide support structures are coated with the cell adhesion molecules. The low dimensional manganese dioxide support structures are either partially, substantially or fully coated with the cell adhesion molecules. The cell adhesion molecules can be applied to the dimensional manganese dioxide support structures by any suitable method. For example, in some embodiments, the cell adhesion molecules can be applied by at least one of coating, spraying, or self-assembly. The self-assembly can occur by any suitable method, such as, for example, electrostatic, hydrophobic or hydrophilic interactions, van der Waals interactions, or hydrogen bonding. The coated low dimensional manganese dioxide support structures are mixed with a cell suspension containing a plurality of cells. This results in a mixture, containing coated low dimensional manganese dioxide support structures and the cells, which have self-assembled into a plurality of spheroids. For example, see FIG. 1A which illustrates a 2-D MnO₂ nanosheet having its surface coated with cell adhesion molecules (represented by wrench-shaped appendages in the left side of FIG. 1A) that have attached thereto cell binding domains, where the nanosheet also contains a therapeutic agent loaded onto its surface (represented by stars). When mixed with the cell suspension, the nanosheet self-assembles into a hybrid spheroid (middle of FIG. 1A). Subsequently the therapeutic agent can be released from the spheroid (right side of FIG. 1A). Any suitable cells can be used in the mixture, as described hereinabove. In some embodiments, for example, the cells are stem cells, endoderm cells, mesoderm cells, ectoderm cells, cancer cells, or a combination thereof. In some embodiments, the cells are at least one of neural stem cells, mesenchymal stem cells, or induced pluripotent stem cells (iPSCs). Any suitable concentration of cells and low dimensional manganese dioxide support structures may be used. In some embodiments, solutions containing the low dimensional manganese dioxide support structures, cells and cell suspension medium have a cell concentration ranging from about 100,000 to about 10 million cells per mL. In some embodiments, solutions containing the low dimensional manganese dioxide support structures, cells and cell suspension medium have a low dimensional manganese dioxide support structure concentration ranging from about 1 ug/mL to about 50 ug/mL.

The cells in the cell suspension can be suspended in any suitable suspension medium. In some embodiments, the cell suspension medium is a natural medium, such as for example, biological fluids (for example, plasma, serum, lymph, human placental cord serum, or amniotic fluid), tissue extracts (for example, extract of liver, spleen, tumors, leucocytes and bone marrow, or extract of bovine or chick embryo), clots (for example, coagulants or plasma clots), Platelette lysates or balanced salt solutions (for example, PBS (phosphate-buffered saline), DPS (Dulbecco's phosphate buffered salines), HBSS (Hanks' balanced salt solutions) or EBSS (Earle's balanced salt solutions)). In some embodiments, the cell suspension medium is an artificial medium, such as for example, a basal medium (for example, MEM (Minimum Essential Medium), Neurobasal Medium, or DMEM (Dulbecco's Modified Eagle's Medium)), or complex medium (for example, RPMI-1640 (Roswell Park Memorial Institute 1640 Medium), DMEM/F12 or IMDM (Iscove's Modified Dulbecco's Media)). Any suitable concentration of cell suspension medium may be used. In some embodiments, solutions containing the low dimensional manganese dioxide support structures, cells and cell suspension medium have a cell suspension medium concentration of from about ug/mL to about 1 mg/mL.

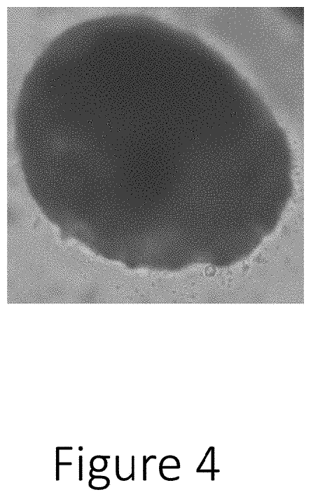

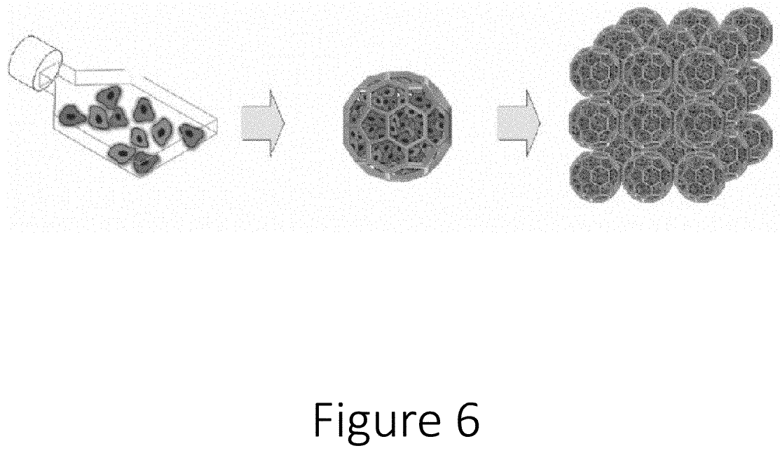

The self-assembly of the low dimensional manganese dioxide support structures and the cells into the plurality of spheroids takes place rapidly, which enables more timely treatment of patients. In some embodiments, the self-assembly takes no more than 5 minutes. In some embodiments, the spheroid self-assembly takes a period of time of from about 1 minute to about 5 minutes. If left for a longer period of time, the spheroids will continue to grow in size, and after a sufficient amount of time, the spheroids will form at least one organoid (see FIG. 4 showing a beating cardiac tissue organoid produced from cardiac tissue, and FIG. 6 showing a schematic diagram where culturing produces a plurality of spheroids combining to form larger spheroids that can become organoids). The organoids are artificially grown masses of cells or tissue that are each a miniaturized or simplified version of an organ, and that exhibit realistic micro-anatomy and physiologic relevance. Organoid formation requires the differentiation of stem cells into multiple cell lineages of a tissue or organ, and extended culturing time is needed, thus the cells used to make the organoids typically include at least one of organ specific adult-stem cells (ASCs) or pluripotent stem cells (PSCs). In addition, complex cocktails of growth factors and small molecules are necessary for organoid growth and development including but not limited to BDNF, GDNF, Sodium Pyruvate, IWR-1, SB431542, Beta-Mercaptoethanol, bfgf, egf, noggin. Typically, a sufficient quantity of cells to form at least one organoid is initially assembled within a period of time of no more than 24 hours. The differentiation of the organoid cells typically takes a longer period of time, for example, up to a few weeks. In some embodiments, the organoids are initially assembled within from 20 minutes to 24 hours. In some embodiments, the spheroids or organoids are formed 1-2 orders faster than those formed by conventional 3D cell culture systems. As noted above, the low dimensional manganese dioxide support structures degrade over time. Thus, after a period of time, the low dimensional manganese dioxide support structures in the spheroids or organoids will begin to degrade. In some embodiments, by the time the organoids have fully formed, the low dimensional manganese dioxide support structures are partially degraded. In some embodiments, by the time the organoids have fully formed, the low dimensional manganese dioxide support structures are either substantially or fully degraded. The rate of degradation of the low dimensional manganese dioxide support structures can be tuned as described hereinabove.

Likewise, the size of the spheroids or organoids can be tuned. In some embodiments, the size of the spheroids or organoids is tuned by controlling the ratio of cells to zero-dimensional, one-dimensional or two-dimensional manganese dioxide support structures, the concentration of the cells, or the concentration of the zero-dimensional, one-dimensional or two-dimensional manganese dioxide support structures, or a combination thereof.

In some embodiments, at least one agent in loaded onto the low dimensional manganese dioxide support structures before they are mixed with the cell suspension. In one embodiment, at least one therapeutic agent is loaded on the low dimensional manganese dioxide support structures. Any suitable therapeutic agent can be used, as described hereinabove. The therapeutic agent can be loaded onto the low dimensional manganese dioxide support structures by any suitable application method, such as for example coating, spraying, and mixing. The therapeutic agent can be loaded onto the low dimensional manganese dioxide support structures before, concurrently with, or after the cell adhesion molecules are applied to the low dimensional manganese dioxide support structures. In some embodiments, the rate of delivery of the therapeutic agent is controlled by tuning the rate of degradation of the low dimensional manganese dioxide support structures. Loading the therapeutic agent onto the low dimensional manganese dioxide support structures prior to mixing the support structures with the cell suspension facilitates deep drug delivery into the spheroids or organoids in a degradation-dependent manner, which provides substantially enhanced control over cell behaviors of cells located inside the spheroids or organoids, and avoids undesired necrotic core, even in large-sized spheroids or organoids.

In some embodiments, the mixture containing the biodegradable scaffolding material is subjected to vacuum filtration, which is a method that is well known in the art, in order to isolate the spheroids or the organoids. In some embodiments, the mixture containing the biodegradable scaffolding material is centrifuged before being subjected to vaccum filtration.

As used herein, the term “antibody” (Ab) is used in the broadest sense, and specifically can include any immunoglobulin, whether natural, or partly, or wholly synthetically produced, including, but not limited to monoclonal antibodies, polyclonal antibodies, multispecific antibodies (for example, bispecific antibodies and polyreactive antibodies), and antibody fragments. Thus, the term “antibody,” as used in any context within this specification, is meant to include, but not be limited to, any specific binding member, immunoglobulin class and/or isotype (e.g., IgG1, IgG2a, IgG2b, IgG3, IgG4, IgM, IgA1, IgA2, IgD, and IgE), and biologically relevant fragment, or specific binding member thereof, including, but not limited to, Fab, F(ab′)₂, scFv (single chain or related entity) and (scFv)₂.

As used herein, the term “biologic drug” can refer to an antibody coupled to a therapeutic agent, or antibody coupled to a therapeutic drug or agent, via a degradable or cleavable linker, e.g. an antibody-drug conjugate (ADC). ADC technology is known in the art, and is covered in, for example, Beck et al. Nature Reviews Drug Discovery 16, 315-337 (2017), hereby incorporated by reference in its entirety.

As used herein, the term “cytokine” can refer to any substances secreted by cells of the immune system that have an effect on other cells, including both anti-inflammatory and pro-inflammatory cytokines. Exemplary cytokines include, but are not limited to, those in the IL-1 superfamily, TNF superfamily, interferons, chemokines, and IL-6 superfamily, as well receptors of any cytokines.

As used herein, the term “nucleic acid,” can refer to a polymer composed of a multiplicity of nucleotide units (ribonucleotide, deoxyribonucleotide, or related structural variants) linked via phosphodiester bonds, including but not limited to, DNA or RNA. The term encompasses sequences that include any of the known base analogs of DNA and RNA. Examples of a nucleic acid include, and are not limited to, mRNA, miRNA, tRNA, rRNA, snRNA, siRNA, dsRNA, cDNA and DNA/RNA hybrids. Nucleic acids can be single stranded or double stranded, or can contain portions of both double stranded and single stranded sequences. The nucleic acid can be DNA, both genomic and cDNA, RNA, or a hybrid, where the nucleic acid can contain combinations of deoxyribo- and ribo-nucleotides, and combinations of bases including uracil (U), adenine (A), thymine (T), cytosine (C), guanine (G), and their derivative compounds. Nucleic acids can be obtained by chemical synthesis methods or by recombinant methods. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof.

As used herein, the term “peptide” can refer to peptide compounds containing two or more amino acids linked by the carboxyl group of one amino acid to the amino group of another, to form an amino acid sequence. Peptides can be purified and/or isolated from natural sources or prepared by recombinant or synthetic methods. A peptide can be a linear peptide or a cyclopeptide, i.e. cyclic, including bicyclic. A “cyclic peptide” or “cyclopeptide,” as used herein, can refer to a peptide having at least one internal bond attaching nonadjacent amino acids of the peptide. A “bicyclic peptide” can have at least two internal bonds forming a cyclopeptide.

As used herein, the term “patient” or “subject” can be used interchangeably. A “subject” can refer to a biological system to which a treatment can be administered. A biological system can include, for example, an individual cell, a set of cells (e.g., a cell culture), an organ, a tissue, or a multi-cellular organism. A “patient” or “subject” can refer to a human patient or a non-human patient.

As used herein, the term “treating” or “treatment” of a disease refers to executing a protocol, which can include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. The terms “prevent” or “preventing” refer to prophylactic and/or preventative measures, wherein the object is to prevent, or slow down the targeted pathologic condition or disorder. In addition, “treating” or “treatment” does not require complete alleviation of signs or symptoms, does not require a cure, and specifically includes protocols that have only a marginal effect on the patient.

As used herein, and in the appended claims, the singular forms “a”, “and” and “the” include plural references, unless the context clearly dictates otherwise.

The term “about” refers to a range of values which would not be considered by a person of ordinary skill in the art as substantially different from the baseline values. For example, the term “about” can refer to a value that is within 20%, 15%, 10%, 9%, 8%, 7%, 6%, 5%, 4%, 3%, 2%, 1%, 0.5%, 0.1%, 0.05%, or 0.01% of the stated value, as well as values intervening such stated values. Context will dictate which value, or range of values, the term “about” can refer to in any given instance, throughout this disclosure.

Where a value of ranges is provided, it is understood that each intervening value, between the upper and lower limit of that range, and any other stated or intervening value in that stated range, is encompassed within the invention. The upper and lower limits of these smaller ranges, which can independently be included in the smaller ranges, is also encompassed within the invention, subject to any specifically excluded limit in the stated range.

Each of the applications and patents cited in this text, as well as each document or reference, patent or non-patent literature, cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference in their entirety. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention.

The following non-limiting examples serve to further illustrate the present invention.

### EXAMPLES

The following examples describe the synthesis and characterization of the biodegradable hybrid nanoscaffold material, as well as their use in stem cell therapy and as a drug delivery platform. Example 1 relates to the materials and methods utilized in Examples 2-6.

### Example 1. Synthesis and Characterization of the Biodegradable Hybrid Nanoscaffold Material

### Synthesis and Characterization of Spheroidal Nanoscaffold Material

Gamma phase manganese dioxide nanosheets with an average size of 20 nm measured by DLS and zeta potential of −35 mV were synthesized. The formation of nanosheets was then characterized using TEM (transmission electron microscopy) and XPS (X-ray photoelectron spectroscopy), confirming the size and composition of the nanosheets. These gamma phase MnO₂ nanosheets had shown the ability to degrade via a redox mechanism and had a high drug/ECM protein loading. The ability of nanosheets to load the common neuronal ECM protein laminin was confirmed. Hybrid spheroids were assembled using iPSC-derived neural stem cells (iPSC-NSC) as the starting cell type. Solutions containing laminin protein, cells, and the nanosheets, and having a cell concentration of 1 million cells per mL and a laminin protein concentration of 25 ug/mL, were mixed, and the solution self-assembled into hybrid spheroids within minutes. The concentration of nanosheets was varied, and it was determined that from 1 ug/mL up to a concentration of 50 ug/mL there was negligible cytotoxicity, so this concentration range was used for future experiments.

### Differentiation Capability

The capability of the spheroidal nanoscafffold material to differentiate into neuronal lineages was then tested. It had been previously demonstrated that cells grown in spheroids have impaired neuronal differentiation compared to cells grown in 2D cultures, so varying concentrations of manganese dioxide nanosheets were compared to untreated neurospheres and 2D cultures. It was shown that the maximum concentration of MnO₂ nanosheets of 50 ug/mL gave the highest increase in neuronal differentiation as measured by qPCR (quantitative polymerase chain reaction) and immunostaining for the neuronal marker Tuj1. It was hypothesized that the improved neuronal differentiation with increasing concentration of nanosheets was due to the control of cell-cell and cell-matrix interactions that drive neuronal differentiation, and therefore, the hypothesis was tested by looking at gene expression of Notch and FAK (focal adhesion kinase) signaling. It was shown that with an increase in MnO₂ concentration, there was a corresponding increase in focal adhesion signaling, a canonical cell matrix interaction which was shown to drive neuronal differentiation, as well as a repression of Notch signaling, which is a cell-cell interaction pathway which was shown to repress neuronal differentiation through lateral inhibition. Given these results, it was concluded that the hybrid scaffold cell assembly was able to guide the neuronal differentiation of stem cells through modulation of cell-cell and cell-matrix interactions.

### Example 2. Controlled Loading, Degradation and Release of Small Molecules for Soluble Signal Transduction

For this study, the drug DAPT (N-[N-(3,5-difluorophenacetyl)-L-alanyl]-S-phenylglycine t-butyl ester), a notch inhibitor, was selected, which had been shown to be highly efficient at inducing neurogenesis. First, the drug loading of DAPT was tested on the surface of the MnO₂ nanosheets using MALDI-TOF (matrix-assisted laser desorption/ionization-time-of-flight mass spectrometer) analysis, and it was confirmed that DAPT could be loaded on the surface of the nanosheets.

Next, the degradation of the nanosheets and the corresponding drug release were studied. MnO2 nanosheets were micro-patterned using soft lithography, and the micro-pattern was characterized using field emission scanning electron microscopy, which showed the clear, dark line pattern of MnO₂. Micropatterened samples were then treated with ascorbic acid, a common bioreductant that can be released by cells, and degradation was shown, as the pattern was no longer visible. This was also characterized by EDX (energy-dispersive X-ray spectroscopy) mapping where a depletion of the Mn peak was seen after addition of ascorbic acid.

The time dependent degradation of the MnO₂ in the hybrid cell spheroids was then tested. The spheroid was cultured for several days, and the supernatant was collected regularly. ICP-MS (inductively coupled plasma mass spectrometry) was used to analyze the ionic manganese content, to track the percent degradation of the scaffold. It was shown that approximately 60% of the scaffold was degraded within 5 days in a linear trend. In addition, a fluorescent molecule was loaded as a model drug for tracking purposes, and a time-dependent drug release was shown using fluorescent microscopy.

Lastly, because Mn²⁺ ions are MRI active, they could be used for MRI monitoring of degradation and drug release. A 7-day degradation study was performed, and it was shown that the Mn2+ ions generated correlated with the amount of the fluorescent model drug that was released, showing that the drug was released through the degradation of the scaffold nanosheets. After confirming the drug loading and degradation properties of the scaffold nanosheets and the hybrid spheroids, varying concentrations of DAPT were then loaded on the surface of the nanosheets at thicknesses of from 0 to 50 μM. The spheres were cultured for 7 days and then the neuronal differentiation was analyzed using both qPCR and immunostaining. It was shown that at day 7, a loaded concentration of 5 and 10 μM significantly enhanced the differentiation of IPSC-NSC into neurons as shown by a ca. 1.8 fold increase in mRNA expression of Tuj1, and a significant increase in Tuj1 positive cells, as shown by immunostaining. These cells also showed longer axons and an increase in mature phenotype markers, such as Map2 (microtubule-associated protein 2) and NeuN (neuronal nuclei), as shown by qPCR and immunostaining. The 50 μM condition showed significant cell death and impaired neurogenesis, the 5 μM condition was selected to move forward with future experiments.

### Example 3. In Vivo Delivery of Spheroidal Nanoscaffold Material for the Treatment of Acute Spinal Cord Injury

Neural stem cells have several key advantages for the treatment of spinal cord injury. They not only have the ability to differentiate into the correct host cell type in order to reestablish neuronal circuitry, but they also have the ability to better survive transplantation into a highly inflamed injury site, and help mitigate that inflammation. In this example, the ability of the scaffold material system to better enhance the survival of neural stem cells after transplantation into the injury site was tested.

An in vitro model of inflammation was used. THP-1 (1-Keto-1,2,3,4-tetrahydrophenanthrene) monocytes were cultured and induced into macrophages. The macrophages were then stimulated using LPS (lipopolysaccharide) to activate their secretion of pro-inflammatory cytokines. This conditioned media was then combined with iPSC-NSC media in a 1:10 ratio. Neurospheres with the MnO₂ scaffold, neurospheres without the MnO₂ scaffold, neurospheres with the growth factors bFGF (basic fibroblast growth factor) and EGF (epidermal growth factor), and neurospheres without bFGF and EGF, were cultured in the combined media and assayed for cell survival. It had been shown that the addition of bFGF and EGF, along with many other growth factors, were able to improve the survival of transplanted stem cells into spinal cord injury. It was shown that the addition of bFGF and EGF, as well as the addition of MnO₂ by itself could improve the survival of the neurospheres after addition of the conditioned media. However, it was seen that when loading the bFGF and EGF on the surface of the nanosheets, the hybrid MnO₂ scaffold-neurospheres showed the greatest survival of >90%. This could possibly be attributed to the combination of physical cue mediated survival, as well as deep delivery of growth factors throughout the neurospheres. These findings make it the ideal solution for aiding the survival of transplanted neural stem cells. A 28GA needle was to be used for injection of the neurospheres into the injury site, therefore control over the size of the neurospheres was important. To this end, PDMS microwells of varying sizes (75, 150, and 300 um) were used in order to template the growth of the hybrid neurospheres. The components, namely 1 million cells, 50 ug of MnO₂ and 25 ug of laminin, for the hybrid neurospheres, were mixed together, and then plated on the microwells in growth media, and allowed to form overnight. The hybrid spheres were then transferred to tissue culture plates in differentiation media, and allowed to differentiate for 7 days, after which they were assayed with immunostaining. It was determined that the 150 um and 300 um spheres showed the best neuronal differentiation, and therefore, the 150 um size spheres were chosen, as they would be easier to inject for the in vivo studies.

Based on the promising ability of the hybrid neurospheres to enhance neuronal differentiation based on soluble and insoluble signalling, surviving inflammatory conditions, and providing MRI monitorable degradation, the hybrid neurospheres were then tested in an in vivo spinal cord injury model. For the studies a T10 hemisection model that only paralyzed one hind limb was utilized using C57B16 mice that were 8 weeks old. During surgery, a hemisection was performed, and then immediately, control single cells (cells with bFGF+EGF+DAPT in solution), control neurospheres (neurospheres with BFGF+EGF+DAPT in solution), and hybrid-neurospheres (neurospheres with MnO₂ and bFGF+EGF+DAPT loaded on the scaffold) were injected. GFP (Green Fluorescent Protein) labeled-iPSC NSCs were used in order to track the cells after transplantation.

First, the number of transplanted cells in the spinal cord after 1 week were studied. It was shown that there was a statistically significant increase in the number of cells present in the spinal cord after 1 week in the hybrid-neurosphere condition, compared to both neurosphere and single cell injections, thus demonstrating the ability of the hybrid spheroid system to aid in the transplantation and survival of the transplanted cells.

The differentiation of the transplanted cells using Tuj1 immunostaining was then evaluated. It was shown that a significantly higher percentage of GFP+ cells were also Tuj1+ in the hybrid-neurosphere condition. This showed the ability of hybrid-neurospheres to guide the differentiation of stem cells into neurons at a higher rate than single cells injection or neurosphere injections.

In a separate experiment, animals were subjected to a T10 hemisection model that only paralyzed one hind limb was utilized using C57B16 mice that were 8 weeks old. During surgery, a hemisection was performed, and then immediately, no cell treatment (bFGF+EGF+DAPT in solution), control single cells (cells with bFGF+EGF+DAPT in solution), control neurospheres (neurospheres with BFGF+EGF+DAPT in solution), and hybrid-neurospheres (neurospheres with MnO₂ and bFGF+EGF+DAPT loaded on the scaffold) were injected. Mice were subject to blinded BMS scoring at day 0 right after surgery, day 2, 4, 7, 14, 21, and 28 where locomotor function was tested using the BMS scoring system, which is commonly used to evaluate locomotor function following SCI or other trauma.

Taken together, this test showed the ability of the hybrid cell spheroids (hybrid-neurospheres) to enhance both the survival and differentiation of transplanted stem cells in spinal cord injury.

The foregoing examples and descriptions of embodiments should be taken as illustrating, rather than as limiting the present invention as defined by the claims. As will be readily appreciated, numerous variations and combinations of the features set forth above can be utilized without departing from the present invention, as set forth in the claims. Such variations are not regarded as a departure from the scope of the invention, and all such variations are intended to be included within the scope of the following claims. All references cited herein are incorporated by reference in their entireties.

What is claimed is:

1. A biodegradable scaffolding material comprising a plurality of at least one of zero-dimensional, one-dimensional or two-dimensional manganese dioxide support structures, and a plurality of cells,
wherein the zero-dimensional, one-dimensional or two-dimensional manganese dioxide support structures each have a surface, and upon each surface is a coating comprising a plurality of cell adhesion molecules;
wherein the zero-dimensional, one-dimensional or two-dimensional manganese dioxide support structures define a structure comprising a plurality of interstices, and the plurality of cells are disposed around and between the zero-dimensional, one-dimensional or two-dimensional manganese dioxide support structures and through the zero-dimensional, one-dimensional or two-dimensional manganese dioxide support structure interstices;
wherein the cell adhesion molecules comprise a plurality of cell binding domains, have a binding affinity with the zero-dimensional, one-dimensional or two-dimensional manganese dioxide support structures, and promote the adhesion of the cells to the zero-dimensional, one-dimensional or two-dimensional manganese dioxide support structures; and
wherein together, the support structures and the plurality of cells are configured to self-assemble to form at least one spheroid.

2. The biodegradable scaffolding material of claim 1, wherein the cell adhesion molecules comprise at least one of biopolymers or small molecules,
wherein the small molecules have at least one of a mass of no more than 900 daltons, a molecular weight of no more than 1500, or a size of from about 0.5 nm to about 1.5 nm.

3. The biodegradable scaffolding material of claim 1, wherein the cell binding domains are peptides comprising at least one of an arginine-glycine-aspartic acid (RGD) amino acid sequence, a cationic amine group, an amphiphilic unit, or a combination or two or more thereof.

4. The biodegradable scaffolding material of claim 1, wherein the zero-dimensional, one-dimensional or two-dimensional manganese dioxide support structures comprise at least one of, 0-dimensional nanoparticles, 1-dimensional manganese dioxide nanotubes, 1-dimensional manganese dioxide nanorods, or 2-dimensional manganese dioxide nanosheets.

5. The biodegradable scaffolding material of claim 1, wherein the cells are selected from the group consisting of stem cells, endoderm cells, mesoderm cells, ectoderm cells, cancer cells, and a combination of two or more thereof.

6. The biodegradable scaffolding material of claim 1, wherein the scaffolding material further comprises at least one therapeutic agent selected from the group consisting of a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, diagnostic composition, psychiatric medication, psychoactive compound, and a combination of two or more thereof.

7. A method of making a biodegradable scaffolding material comprising:
(i) providing at least one of a plurality of zero-dimensional, one-dimensional or two-dimensional manganese dioxide support structures;
(ii) applying a plurality of cell adhesion molecules comprising a plurality of cell binding domains to a surface of the zero-dimensional, one-dimensional or two-dimensional manganese dioxide support structures; and
(iii) mixing the zero-dimensional, one-dimensional or two-dimensional manganese dioxide support structures with a cell suspension comprising a plurality of cells, so that the support structures, cell adhesion molecules and cells self-assemble in the suspension to form a mixture containing a plurality of spheroids;
wherein the spheroids self-assemble within a time period of about 1 minute to about 5 minutes.

8. The method of claim 7, wherein the mixture is cultured for a period of time sufficient for the spheroid mixture to further form at least one organoid;
wherein the at least one organoid is formed within a time period of no more than 24 hours.

9. The method of claim 7, wherein the plurality of cell adhesion molecules are applied to the surface of the zero-dimensional, one-dimensional or two-dimensional manganese dioxide support structures by a method selected from the group consisting of coating; spraying; and self-assembly via at least one of electrostatic interactions, hydrophobic interactions, hydrophilic interactions, van der waals interactions, hydrogen bonding, or a combination of two or more thereof.

10. The method of claim 7, wherein the cells are selected from the group consisting of stem cells, endoderm cells, mesoderm cells, ectoderm cells, cancer cells, and a combination of two or more thereof.

11. The method of claim 7, wherein the size of the spheroid or organoid is tuned by controlling at least one of
the ratio of cells to zero-dimensional, one-dimensional or two-dimensional manganese dioxide support structures,
the concentration of the cells, or
the concentration of the zero-dimensional, one-dimensional or two-dimensional manganese dioxide support structures.

12. The method of claim 7, further including the step of loading at least one therapeutic agent onto the zero-dimensional, one-dimensional or two-dimensional manganese dioxide support structures, wherein the therapeutic agent is selected from the group consisting of a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, diagnostic composition, psychiatric medication, psychoactive compound, and a combination of two or more thereof.

13. The method of claim 12, wherein the rate of delivery of the therapeutic agent is controlled by tuning the rate of biodegradation of the zero-dimensional, one-dimensional or two-dimensional manganese dioxide support structure.

14. The method of claim 13, wherein the rate of biodegradation of the zero-dimensional, one-dimensional or two-dimensional manganese dioxide support structure is tuned by controlling at least one of: the porosity of the scaffolding material, the thickness of the scaffolding material, the aspect ratio of the scaffolding material, or the cell density.

15. The method of claim 7, wherein the rate at which the biodegradable scaffolding material is degraded in vivo is measured by detecting the rate of release of Mn⁺² ions from the biodegradable scaffolding material.

16. The method of claim 7, wherein the zero-dimensional, one-dimensional or two-dimensional manganese dioxide support structures comprise at least one of, 0-dimensional nanoparticles, 1-dimensional manganese dioxide nanotubes, 1-dimensional manganese dioxide nanorods, or 2-dimensional manganese dioxide nanosheets.

17. The method of claim 7, wherein the cell adhesion molecules comprise at least one of biopolymers or small molecules,
wherein the small molecules have at least one of a mass of no more than 900 daltons, a molecular weight of no more than 1500, or a size of from about 0.5 nm to about 1.5 nm.

18. The method of claim 7, wherein the cell binding domains are peptides comprising at least one of an arginine-glycine-aspartic acid (RGD) amino acid sequence, a cationic amine group, an amphiphilic unit, or a combination or two or more thereof.

19. The method of claim 7, further comprising the step of vacuum filtering the resultant mixture to isolate the biodegradable scaffolding material, and optionally, further comprising the step of centrifuging the resultant mixture prior to vacuum filtering.

20. A method of treating a disease or disorder in a subject, comprising surgically implanting or injecting the biodegradable scaffolding material according to claim 1 into a subject in need thereof.
